(19) United States
(12) Patent Application Publication
(10) Pub. No.: US 2026/0130580 A1
(43) Pub. Date: May 14, 2026

(54) MEDICAL SUPPORT DEVICE, ENDOSCOPE APPARATUS, MEDICAL SUPPORT METHOD, AND PROGRAM

(71) Applicant: FUJIFILM Corporation, Tokyo (JP)

(72) Inventor: Seiya TAKENOUCHI, Kanagawa (JP)

(73) Assignee: FUJIFILM Corporation, Tokyo (JP)

(21) Appl. No.: 19/440,655

(22) Filed: Jan. 6, 2026

Related U.S. Application Data
(63) Continuation of application No. PCT/JP2024/012108, filed on Mar. 26, 2024.

(30) Foreign Application Priority Data
Jul. 21, 2023 (JP) 2023-119509

(51) Int. Cl. A61B 1/005; A61B 1/00

(52) U.S. Cl. CPC A61B 1/0051; A61B 1/00006; A61B 1/000094; A61B 1/000096

(57) ABSTRACT

A medical support device includes a processor. The processor acquires a plurality of frames obtained in time series by imaging, with a camera, an inside of a luminal organ into which a hemostat is inserted, the plurality of frames showing stages of a hemostasis treatment using the hemostat. Further, the processor outputs hemostasis period identification information that enables determination that a period from when the stages shown in the plurality of acquired frames change from a hemostasis preparation stage using the hemostat to a hemostasis start stage using the hemostat until a certain period of time elapses is a hemostasis period during which the hemostasis treatment is performed.

### CROSS-REFERENCE TO RELATED APPLICATIONS

This application is a continuation application of International Application No. PCT/JP2024/012108 filed March 26, 2024, the disclosure of which is incorporated herein by reference in its entirety. Further, this application claims priority from Japanese Patent Application No. 2023-119509, filed July 21, 2023, the disclosure of which is incorporated herein by reference in its entirety.

### DESCRIPTION

### BACKGROUND

### 1. Technical Field

The present disclosure relates to a medical support device, an endoscope apparatus, a medical support method, and a program.

### 2. Related Art

JP6875038B discloses an approach in which images of a plurality of frames constituting a video obtained from an endoscope camera are analyzed to determine whether a hemostatic instrument is included in a surgical field video. Further, the technique described in JP6875038B performs control such that a partial moving image is played back when the surgical field image does not include the hemostatic instrument, and the partial moving image is not played back when the hemostatic instrument appears in the surgical field video.

JP2016-062488A describes an approach in which a treatment tool used in the body of a patient during an endoscopic examination is detected by image processing from frame images included in a moving image obtained by capturing images of the inside of the body of the patient with an endoscope. JP2016-062488A further describes that a treatment tool to be detected includes a hemostat and that the treatment tool may be detected using color information of a sheath. Furthermore, JP2016-062488A describes that, of an endoscopic video, images during a period from when the treatment tool enters the frame until the treatment tool exits the frame are recorded as recorded data.

WO2022/181714A describes an approach in which a trained model is used to recognize a plurality of steps of surgery or identify instruments such as forceps from predetermined consecutive frames in surgical images (a moving image or still images continuously captured in a time-series manner) obtained in endoscopic surgery or similar procedures.

JPWO2019-181432A1 describes a surgical support system that predicts the occurrence of dangerous conditions by using a trained determination device. JPWO2019-181432A1 further describes an approach for generating training data used for learning of the determination device, in which features of hemostatic surgical instruments are detected from surgical images obtained from an endoscope camera or similar devices to detect that hemostasis is being performed.

JP2020-081332A discloses an endoscope-related business system that performs, for example, generation of a report related to an endoscopic examination. JP2020-081332A describes an approach in which a treatment recognition unit included in the endoscope-related business system recognizes forceps used for biopsies (biopsy forceps) by using one or a plurality of endoscopic images to recognize the presence or absence of biopsies. JP2020-081332A further describes that recognition of a type of treatment also includes hemostasis in addition to biopsies.

### SUMMARY

An embodiment according to the present disclosure provides a medical support device, an endoscope apparatus, a medical support method, and a program that allow a user or others to grasp a period of time during which hemostasis is performed using a hemostat.

A first aspect according to the present disclosure is a medical support device including a processor. The processor is configured to acquire a plurality of frames obtained in time series by imaging, with a camera, an inside of a luminal organ into which a hemostat is inserted, the plurality of frames showing stages of a hemostasis treatment using the hemostat; and output hemostasis period identification information that enables determination that a period from when the stages shown in the plurality of acquired frames change from a hemostasis preparation stage using the hemostat to a hemostasis start stage using the hemostat until a certain period of time elapses is a hemostasis period during which the hemostasis treatment is performed.

A second aspect according to the present disclosure is the medical support device according to the first aspect, in which the processor is configured to perform an object recognition process based on the plurality of acquired frames to detect a change from the hemostasis preparation stage to the hemostasis start stage, and the hemostasis period is a period from when the change is detected by the processor until the certain period of time elapses.

A third aspect according to the present disclosure is the medical support device according to the second aspect, in which the object recognition process is implemented by using a model obtained by machine learning performed on a neural network.

A fourth aspect according to the present disclosure is the medical support device according to any one of the first to third aspects, in which the hemostat has a sheath, the plurality of frames include a first frame and a second frame, the first frame is a frame that shows the sheath, the second frame is a frame that does not show the sheath, the hemostasis preparation stage is a stage in which the first frame is obtained, and the hemostasis start stage is a stage in which the second frame is obtained subsequent to the first frame.

A fifth aspect according to the present disclosure is the medical support device according to any one of the first to third aspects, in which the hemostat has a sheath and a clip pin, the plurality of frames include a first frame and a second frame, the first frame is a frame that shows the sheath, the second frame is a frame that shows the clip pin, the hemostasis preparation stage is a stage in which the first frame is obtained, and the hemostasis start stage is a stage in which the second frame is obtained subsequent to the first frame.

A sixth aspect according to the present disclosure is the medical support device according to any one of the first to third aspects, in which the hemostat has a sheath and a clip pin, the plurality of frames include a first frame and a second frame, the first frame is a frame that shows the sheath, the second frame is a frame that shows the clip pin and does not show the sheath, the hemostasis preparation stage is a stage in which the first frame is obtained, and the hemostasis start stage is a stage in which the second frame is obtained subsequent to the first frame.

A seventh aspect according to the present disclosure is the medical support device according to any one of the fourth to sixth aspects, in which the plurality of frames further include a third frame, the third frame is a frame that shows the sheath, and the certain period of time is reset in a case where the third frame is obtained before the certain period of time elapses after a change from the hemostasis preparation stage to the hemostasis start stage.

An eighth aspect according to the present disclosure is the medical support device according to any one of the first to seventh aspects, in which hemostasis treatment related information regarding the hemostasis treatment within the hemostasis period is stored in a storage area.

A ninth aspect according to the present disclosure is the medical support device according to the eighth aspect, in which the hemostasis treatment related information includes the hemostasis period identification information or a portion of the hemostasis period identification information.

A tenth aspect according to the present disclosure is the medical support device according to the ninth aspect, in which the portion includes start timing information that enables identification of a start timing of the hemostasis period and/or end timing information that enables identification of an end timing of the hemostasis period.

An eleventh aspect according to the present disclosure is the medical support device according to any one of the eighth to tenth aspects, in which the hemostasis treatment related information includes hemostasis site related information regarding a site where the hemostasis treatment has been performed in the luminal organ.

A twelfth aspect according to the present disclosure is the medical support device according to the eleventh aspect, in which the hemostasis site related information includes lesion-related information regarding a lesion present in the site where the hemostasis treatment has been performed.

A thirteenth aspect according to the present disclosure is the medical support device according to the twelfth aspect, in which the lesion-related information includes a lesion image obtained by imaging the lesion with the camera.

A fourteenth aspect according to the present disclosure is the medical support device according to any one of the eleventh to thirteenth aspects, in which the hemostasis site related information includes a post-hemostasis image obtained by imaging, with the camera, the site where the hemostasis treatment has been performed.

A fifteenth aspect according to the present disclosure is the medical support device according to any one of the eleventh to fourteenth aspects, in which the hemostasis site related information includes a pre-hemostasis image obtained by imaging, with the camera, a pre-hemostasis condition of the site where the hemostasis treatment has been performed.

A sixteenth aspect according to the present disclosure is the medical support device according to any one of the eighth to fifteenth aspects, in which the hemostasis treatment related information includes frequency information that enables identification of a number of times the hemostasis treatment has been performed in the luminal organ.

A seventeenth aspect according to the present disclosure is the medical support device according to any one of the first to sixteenth aspects, in which the certain period of time is a period of time statistically determined as a time required for the hemostasis treatment to be performed once using the hemostat.

An eighteenth aspect according to the present disclosure is an endoscope apparatus including the medical support device according to any one of the first to seventeenth aspects and the camera.

A nineteenth aspect according to the present disclosure is a medical support method including acquiring a plurality of frames obtained in time series by imaging, with a camera, an inside of a luminal organ into which a hemostat is inserted, the plurality of frames showing stages of a hemostasis treatment using the hemostat; and outputting hemostasis period identification information that enables determination that a period from when the stages shown in the plurality of acquired frames change from a hemostasis preparation stage using the hemostat to a hemostasis start stage using the hemostat until a certain period of time elapses is a hemostasis period during which the hemostasis treatment is performed.

A twentieth aspect according to the present disclosure is a program for causing a computer to execute processing including acquiring a plurality of frames obtained in time series by imaging, with a camera, an inside of a luminal organ into which a hemostat is inserted, the plurality of frames showing stages of a hemostasis treatment using the hemostat; and outputting hemostasis period identification information that enables determination that a period from when the stages shown in the plurality of acquired frames change from a hemostasis preparation stage using the hemostat to a hemostasis start stage using the hemostat until a certain period of time elapses is a hemostasis period during which the hemostasis treatment is performed.

### BRIEF DESCRIPTION OF THE DRAWINGS

Exemplary embodiments according to the technique of the present disclosure will be described in detail based on the following figures, wherein:

FIG. 1 is a conceptual diagram illustrating an example of an aspect in which an endoscope apparatus is used;

FIG. 2 is a conceptual diagram illustrating an example overall configuration of the endoscope apparatus;

FIG. 3 is a block diagram illustrating an example hardware configuration of an electric system of the endoscope apparatus;

FIG. 4 is a block diagram illustrating an example of main functions according to an embodiment of a processor included in a medical support device, and an example of information stored in storage;

FIG. 5 is a conceptual diagram illustrating an example of the content of a process by a recognition unit and a control unit;

FIG. 6 is a graph (i.e., a histogram) illustrating an example of the relationship between the number of frames required for a single hemostasis treatment using a clip pin to be completed in an endoscopic examination (i.e., the number of frames captured by a camera during a period from the start to the end of a single hemostasis treatment using a clip pin) and the number of times a hemostasis treatment has been performed using a hemostat;

FIG. 7 is a conceptual diagram illustrating an example of the content of a process in which the control unit identifies a hemostasis treatment period;

FIG. 8 is a conceptual diagram illustrating an example of the content of a process performed by the control unit in a case where a sheath appears in a frame before a hemostasis treatment time elapses after a change from a hemostasis preparation stage to a hemostasis start stage;

FIG. 9 is a flowchart illustrating an example of the flow of a medical support process;

FIG. 10 is a conceptual diagram illustrating an example of an aspect in which hemostasis treatment related information is stored in a storage area;

FIG. 11 is a conceptual diagram illustrating an example of the content of hemostasis site related information; and

FIG. 12 is a conceptual diagram illustrating an example of a series of processes in which a computer issues a processing execution request to an external device via a network, the external device executes a process in accordance with the processing execution request, and the computer receives a processing result from the external device.

### DETAILED DESCRIPTION

The following describes an example of an embodiment of a medical support device, an endoscope apparatus, a medical support method, and a program according to the present disclosure with reference to the accompanying drawings.

First, terms used in the following description will be described.

CPU is an abbreviation for "Central Processing Unit". GPU is an abbreviation for "Graphics Processing Unit". GPGPU is an abbreviation for "General-Purpose computing on Graphics Processing Units". APU is an abbreviation for "Accelerated Processing Unit". TPU is an abbreviation for "Tensor Processing Unit". RAM is an abbreviation for "Random Access Memory". NVM is an abbreviation for "Non-volatile memory". EEPROM is an abbreviation for "Electrically Erasable Programmable Read-Only Memory". ASIC is an abbreviation for "Application Specific Integrated Circuit". PLD is an abbreviation for "Programmable Logic Device". FPGA is an abbreviation for "Field-Programmable Gate Array". SoC is an abbreviation for "System-on-a-chip". SSD is an abbreviation for "Solid State Drive". USB is an abbreviation for "Universal Serial Bus". HDD is an abbreviation for "Hard Disk Drive". EL is an abbreviation for "Electro-Luminescence". CMOS is an abbreviation for "Complementary Metal Oxide Semiconductor". CCD is an abbreviation for "Charge Coupled Device". AI is an abbreviation for "Artificial Intelligence". BLI is an abbreviation for "Blue Light Imaging". LCI is an abbreviation for "Linked Color Imaging". I/F is an abbreviation for "Interface". SSL is an abbreviation for "Sessile Serrated Lesion". LAN is an abbreviation for "Local Area Network". WAN is an abbreviation for "Wide Area Network". 5G is an abbreviation for "5th Generation Mobile Communication System".

In the following description, a processor with a reference numeral (hereinafter, simply referred to as a "processor") may be a single arithmetic device or a combination of multiple arithmetic devices. The processor may be a single type of arithmetic device or a combination of multiple types of arithmetic devices. An example of an arithmetic device is a CPU, a GPU, a GPGPU, an APU, a TPU, or the like.

In the following description, a memory with a reference numeral is at least one memory (e.g., volatile memory) such as a RAM that temporarily stores information, and is used as a work memory by the processor.

In the following description, a storage with a reference numeral is one or a plurality of non-volatile storage devices that store various programs, various parameters, and the like. An example of a non-volatile storage device is flash memory, a magnetic disk, magnetic tape, or the like. Other examples of storage include cloud storage.

In the following embodiments, an external I/F with a reference numeral controls the exchange of various types of information between multiple interconnected devices. An example of the external I/F is a USB interface or the like. A communication I/F including a communication processor, an antenna, and the like may be used as the external I/F. The communication I/F controls communication between multiple computers. An example of a communication standard applied to the communication I/F is a wireless communication standard such as 5G, Wi-Fi (registered trademark), or Bluetooth (registered trademark).

In the following embodiments, "A and/or B" is synonymous with "at least one of A or B". That is, "A and/or B" means only A, only B, or a combination of A and B. In this specification, furthermore, a concept similar to that of "A and/or B" is applied also to the expression of three or more matters in combination with "and/or".

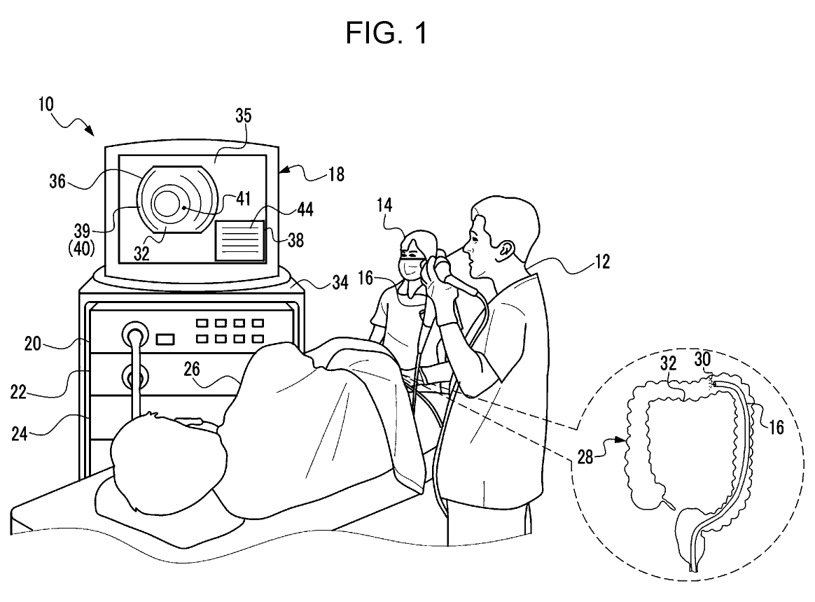

FIG. 1 is a conceptual diagram illustrating an example of an aspect in which an endoscope apparatus 10 is used. As illustrated in FIG. 1, the endoscope apparatus 10 is used by a doctor 12 in an endoscopic examination. The endoscopic examination is assisted by staff such as a nurse 14. In the present embodiment, the endoscope apparatus 10 is an example of an "endoscope apparatus" according to the present disclosure.

The endoscope apparatus 10 is communicably connected to a communication device (not illustrated), and information obtained by the endoscope apparatus 10 is transmitted to the communication device. An example of the communication device is a server and/or a client terminal (e.g., a personal computer, a tablet terminal, and/or the like) that manages various types of information such as electronic medical records. The communication device receives information transmitted from the endoscope apparatus 10 and executes a process using the received information (e.g., a process of storing the information in an electronic medical record or the like).

The endoscope apparatus 10 includes an endoscopic scope 16, a display device 18, a light source device 20, a control device 22, and a medical support device 24.

The endoscope apparatus 10 is a modality for performing a medical examination on a colon 28 included in the body of a subject 26 (e.g., a patient) using the endoscopic scope 16. In the present embodiment, the colon 28 is a target to be observed by the doctor 12 in the endoscopic examination.

The endoscopic scope 16 is used by the doctor 12 and is inserted into a luminal organ of the subject 26. In the present embodiment, the endoscopic scope 16 is inserted into the colon 28 of the subject 26. The endoscope apparatus 10 causes the endoscopic scope 16, which is inserted into the colon 28 of the subject 26, to capture an image of the inside of the colon 28 of the subject 26 and performs various medical treatments on the colon 28 as necessary.

The endoscope apparatus 10 performs imaging of the inside of the colon 28 of the subject 26 to acquire an image indicating the condition of the inside of the colon 28, and outputs the acquired image. In the present embodiment, the endoscope apparatus 10 is an endoscope having an optical imaging function of imaging reflected light obtained by irradiating the inside of the colon 28 with light 30 and capturing light reflected from an intestinal wall 32 of the colon 28.

While the endoscopic examination of the colon 28 is illustrated here, this is merely an example, and the present disclosure is also applicable to an endoscopic examination of a luminal organ such as the esophagus, stomach, duodenum, or trachea.

The light source device 20, the control device 22, and the medical support device 24 are installed in a cart 34. The cart 34 is provided with a plurality of shelves along the vertical direction, and the medical support device 24, the control device 22, and the light source device 20 are installed on the shelves from bottom to top. The display device 18 is installed on top of the cart 34.

The control device 22 controls the entire endoscope apparatus 10. Under the control of the control device 22, the medical support device 24 performs various kinds of image processing on an image obtained by imaging the intestinal wall 32 with the endoscopic scope 16.

The display device 18 displays various types of information including images. An example of the display device 18 is a liquid crystal display, an EL display, or the like. A tablet terminal with a display may be used instead of or together with the display device 18.

The display device 18 displays a screen 35. The screen 35 includes a plurality of display regions. The plurality of display regions are arranged side by side on the screen 35. In the example illustrated in FIG. 1, a first display region 36 and a second display region 38 are depicted as an example of the plurality of display regions. The size of the first display region 36 is larger than the size of the second display region 38. The first display region 36 is used as a main display region, and the second display region 38 is used as a sub-display region. The relationship in size between the first display region 36 and the second display region 38 is not limited to this, and the first display region 36 and the second display region 38 may have any relationship in size so as to fit in the screen 35.

The first display region 36 displays an endoscopic moving image 39. The endoscopic moving image 39 is a moving image acquired by imaging the intestinal wall 32 with the endoscopic scope 16 in the colon 28 of the subject 26. In the example illustrated in FIG. 1, a moving image in which the intestinal wall 32 appears is depicted as an example of the endoscopic moving image 39.

The intestinal wall 32 appearing in the endoscopic moving image 39 includes at least one lesion 41 (e.g., in the example illustrated in FIG. 1, one lesion 41) as a region of interest (i.e., an observation target region) to be gazed at by the doctor 12, and the doctor 12 can visually recognize the condition of the intestinal wall 32 including the lesion 41 through the endoscopic moving image 39. In the present embodiment, the lesion 41 is an example of a "lesion" according to the present disclosure.

There are various types of lesions 41, and the types of lesions 41 include, for example, neoplastic polyps and non-neoplastic polyps. The types of the neoplastic polyps include, for example, adenomatous polyps (e.g., SSL). The types of the non-neoplastic polyps include, for example, hamartoma polyps, hyperplastic polyps, and inflammatory polyps. The types exemplified here are types expected in advance as possible types of the lesion 41 when the endoscopic examination is performed on the colon 28, and the type of lesion differs depending on the organ on which the endoscopic examination is performed.

The image displayed in the first display region 36 is one frame 40 included in a moving image configured to include a plurality of frames 40 along a time series. That is, the first display region 36 displays the plurality of frames 40 along a time series at a predetermined frame rate (e.g., several tens of frames/second). In the present embodiment, the frames 40 are an example of "frames" according to the present disclosure.

An example of the moving image displayed in the first display region 36 is a live-view moving image or the like. The live-view moving image is merely an example, and a moving image displayed after being temporarily stored in memory or the like, such as a post-view moving image, may be used. Alternatively, each frame included in a moving image for recording, which is stored in memory or the like, may be played back and displayed on the screen 35 (e.g., the first display region 36) as the endoscopic moving image 39.

On the screen 35, the second display region 38 is present outside the first display region 36. In the example illustrated in FIG. 1, the second display region 38 is adjacent to the first display region 36 and is displayed in the right portion of the screen 35 when viewed from the front. The second display region 38 may be displayed at any position different from the first display region 36, but is preferably displayed at a position that enables comparison with the endoscopic moving image 39 displayed in the first display region 36.

The second display region 38 displays medical information 44, which is information related to medical treatment. Examples of the medical information 44 include information and the like for assisting the doctor 12 in making a medical determination or the like. A first example of the information and the like for assisting the doctor 12 in making a medical determination or the like is various types of visible information related to the subject 26 into which the endoscopic scope 16 is inserted (such as the name, gender, medication, medical history, blood pressure value, heart rate, and/or the like). A second example of the information and the like for assisting the doctor 12 in making a medical determination or the like is visible information such as text and/or images obtained by performing processing using AI on the endoscopic moving image 39 (e.g., a feature map and/or information obtained by processing the feature map).

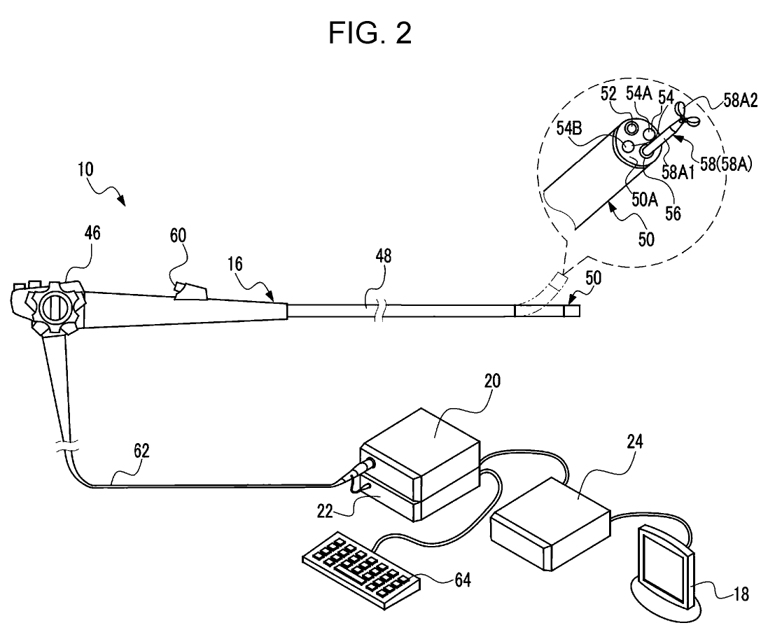

FIG. 2 is a conceptual diagram illustrating an example overall configuration of the endoscope apparatus 10. As illustrated in FIG. 2, the endoscopic scope 16 includes an operation section 46 and an insertion section 48. The insertion section 48 partially bends in response to the operation section 46 being operated. When the doctor 12 (see FIG. 1) operates the operation section 46, the insertion section 48 is inserted into the colon 28 (see FIG. 1) while bending according to the shape of the colon 28.

The insertion section 48 has a tip portion 50 provided with a camera 52, an illumination device 54, and a treatment tool opening 56. The camera 52 and the illumination device 54 are provided on a tip surface 50A of the tip portion 50. While an example is illustrated here in which the camera 52 and the illumination device 54 are provided on the tip surface 50A of the tip portion 50, this is merely an example. The camera 52 and the illumination device 54 may be provided on a side surface of the tip portion 50 such that the endoscopic scope 16 is configured as a side view endoscope.

The camera 52 is inserted into the body cavity of the subject 26 to image an observation target region. In the present embodiment, the camera 52 performs imaging of the inside of the body of the subject 26 (e.g., the inside of the colon 28) to acquire the endoscopic moving image 39. An example of the camera 52 is a CMOS camera or the like. However, this is merely an example, and the camera 52 may be any other type of camera such as a CCD camera. In the present embodiment, the camera 52 is an example of a "camera" according to the present disclosure.

The illumination device 54 has illumination windows 54A and 54B. The illumination device 54 emits the light 30 (see FIG. 1) through the illumination windows 54A and 54B. Examples of the type of the light 30 to be emitted from the illumination device 54 include visible light (e.g., white light or the like) and invisible light (e.g., near-infrared light or the like). The illumination device 54 further emits special light through the illumination windows 54A and 54B. Examples of the special light include light for BLI and/or light for LCI. The camera 52 performs imaging of the inside of the colon 28 using an optical method, with the inside of the colon 28 irradiated with the light 30 from the illumination device 54.

The treatment tool opening 56 is an opening for allowing a treatment tool 58 to protrude from the tip portion 50. The treatment tool opening 56 is also used as a suction port for sucking blood, bodily waste, and the like, and as a delivery port for delivering a fluid.

The operation section 46 has a treatment tool insertion port 60 formed therein, and the treatment tool 58 is inserted into the insertion section 48 through the treatment tool insertion port 60. The treatment tool 58 passes through the insertion section 48 and protrudes to the outside from the treatment tool opening 56. Examples of the treatment tool 58 include a hemostat 58A, a puncture needle, a high-frequency knife, a snare, a catheter, a guide wire, and a cannula. The example illustrated in FIG. 2 presents a manner in which the hemostat 58A as the treatment tool 58 protrude from the treatment tool opening 56. The hemostat 58A is an example of a "hemostat" according to the present disclosure.

The hemostat 58A is used for hemostasis treatment. Hemostasis treatment is a procedure to stop bleeding from the bleeding site (e.g., an excised area that is bleeding as a result of excising the lesion 41 with a snare, a high-frequency knife, or the like). For example, clip hemostasis is used for hemostasis treatment. Clip hemostasis is a method for stopping bleeding by pinching and pressing a bleeding site (such as a blood vessel or a mucous membrane, for example) without using a high-frequency current or the like. Depending on the size of the bleeding site, the amount of bleeding, and/or the like, the hemostasis treatment may be performed ten or more times on a single lesion 41.

The hemostat 58A has a sheath 58A1 and a clip pin 58A2. The clip pin 58A2 protrudes from the distal end of the sheath 58A1 and is clipped to the bleeding site in accordance with an operation by the doctor 12 using a lever (not illustrated) or the like, thereby stopping bleeding from the bleeding site. When clipped to the bleeding site, the clip pin 58A2 is separated from the sheath 58A1 and remains placed in the colon 28. The clip pin 58A2 clipped to the bleeding site is generally excreted from the colon 28 together with stool after the endoscopic examination.

The endoscopic scope 16 is connected to the light source device 20 and the control device 22 through a universal cord 62. The control device 22 is connected to the medical support device 24 and a reception device 64. The medical support device 24 is also connected to the display device 18. That is, the control device 22 is connected to the display device 18 through the medical support device 24.

Since the medical support device 24 is exemplified here as an external device for extending the functions implemented by the control device 22, an example is illustrated here in which the control device 22 and the display device 18 are indirectly connected through the medical support device 24, although this is merely an example. For example, the display device 18 may be directly connected to the control device 22. In this case, for example, the control device 22 may be mounted with the functions of the medical support device 24, or the control device 22 may be mounted with a function of causing a server (not illustrated) to execute the same process as a process executed by the medical support device 24 (e.g., a medical support process described below) and receiving and using a processing result obtained by the server.

The reception device 64 receives an instruction from the doctor 12 and outputs the received instruction to the control device 22 as an electrical signal. An example of the reception device 64 is a keyboard, a mouse, a touch panel, a foot switch, a microphone, a remote operation device, and/or the like.

The control device 22 controls the light source device 20, transmits and receives various signals to and from the camera 52, and transmits and receives various signals to and from the medical support device 24.

The light source device 20 emits light under the control of the control device 22 and supplies the light to the illumination device 54. The illumination device 54 incorporates a light guide, and the light supplied from the light source device 20 is emitted from the illumination windows 54A and 54B through the light guide. The control device 22 causes the camera 52 to perform imaging, acquires the endoscopic moving image 39 (see FIG. 1) from the camera 52, and outputs the endoscopic moving image 39 to a predetermined output destination (e.g., the medical support device 24).

The medical support device 24 performs various kinds of image processing on the endoscopic moving image 39 input from the control device 22 to support medical treatment (here, as an example, endoscopy). The medical support device 24 outputs the endoscopic moving image 39 on which the various kinds of image processing have been performed to a predetermined output destination (e.g., the display device 18).

While an example has been described in which the endoscopic moving image 39 output from the control device 22 is output to the display device 18 through the medical support device 24, this is merely an example. For example, the control device 22 and the display device 18 may be connected to each other, and the endoscopic moving image 39 on which image processing has been performed by the medical support device 24 may be displayed on the display device 18 through the control device 22.

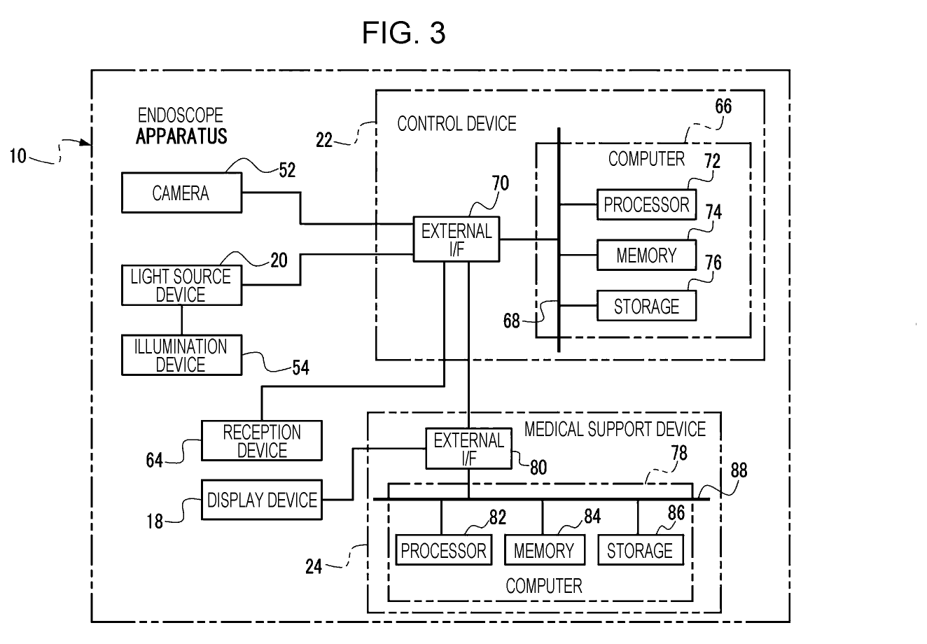

FIG. 3 is a block diagram illustrating an example hardware configuration of an electric system of the endoscope apparatus 10. As illustrated in FIG. 3, the control device 22 includes a computer 66, a bus 68, and an external I/F 70. The computer 66 includes a processor 72, memory 74, and storage 76. The processor 72, the memory 74, the storage 76, and the external I/F 70 are connected to the bus 68. The processor 72 controls the entire control device 22. The memory 74 and the storage 76 are used by the processor 72.

The external I/F 70 handles transmission and reception of various kinds of information between the processor 72 and one or more devices (hereinafter also referred to as "first external devices") external to the control device 22.

The camera 52 is connected to the external I/F 70 as one of the first external devices, and the external I/F 70 handles transmission and reception of various kinds of information between the camera 52 and the processor 72. The processor 72 controls the camera 52 through the external I/F 70. Further, the processor 72 acquires the endoscopic moving image 39 (see FIG. 1), which is obtained by imaging the inside of the colon 28 (see FIG. 1) with the camera 52, through the external I/F 70.

The light source device 20 is connected to the external I/F 70 as one of the first external devices, and the external I/F 70 handles transmission and reception of various kinds of information between the light source device 20 and the processor 72. The light source device 20 supplies light to the illumination device 54 under the control of the processor 72. The illumination device 54 emits the light supplied from the light source device 20.

The reception device 64 is connected to the external I/F 70 as one of the first external devices, and the processor 72 acquires an instruction received by the reception device 64 through the external I/F 70 and executes a process corresponding to the acquired instruction.

The medical support device 24 includes a computer 78 and an external I/F 80. The computer 78 includes a processor 82, memory 84, and storage 86. The processor 82, the memory 84, the storage 86, and the external I/F 80 are connected to a bus 88. In the present embodiment, the medical support device 24 is an example of a "medical support device" according to the present disclosure, the computer 78 is an example of a "computer" according to the present disclosure, and the processor 82 is an example of a "processor" according to the present disclosure.

The hardware configuration of the computer 78 (i.e., the processor 82, the memory 84, and the storage 86) is basically the same as the hardware configuration of the computer 66, and thus the description of the hardware configuration of the computer 78 will be omitted here.

The external I/F 80 handles transmission and reception of various kinds of information between the processor 82 and one or more devices (hereinafter also referred to as "second external devices") external to the medical support device 24.

The control device 22 is connected to the external I/F 80 as one of the second external devices. In the example illustrated in FIG. 3, the external I/F 70 of the control device 22 is connected to the external I/F 80. The external I/F 80 handles transmission and reception of various kinds of information between the processor 82 of the medical support device 24 and the processor 72 of the control device 22. For example, the processor 82 acquires the endoscopic moving image 39 (see FIG. 1) from the processor 72 of the control device 22 through the external I/Fs 70 and 80, and performs various kinds of image processing on the acquired endoscopic moving image 39.

The display device 18 is connected to the external I/F 80 as one of the second external devices. The processor 82 controls the display device 18 through the external I/F 80 to display various kinds of information (e.g., the endoscopic moving image 39 and the like on which the various kinds of image processing have been performed) on the display device 18.

In an endoscopic examination, after the lesion 41 is excised, a hemostasis treatment is performed to stop bleeding from the bleeding site (i.e., the area from which the lesion 41 is excised) with the hemostat 58A. After the hemostasis treatment is completed, the doctor 12 determines the necessity of a subsequent medical treatment (e.g., a further hemostasis treatment) or determines the timing at which the medical treatment is to be performed next. Accordingly, in an endoscopic examination, it is important for the doctor 12 to accurately determine whether a hemostasis treatment is currently being performed (in particular, whether the hemostasis treatment has been completed) in order to implement an appropriate medical treatment.

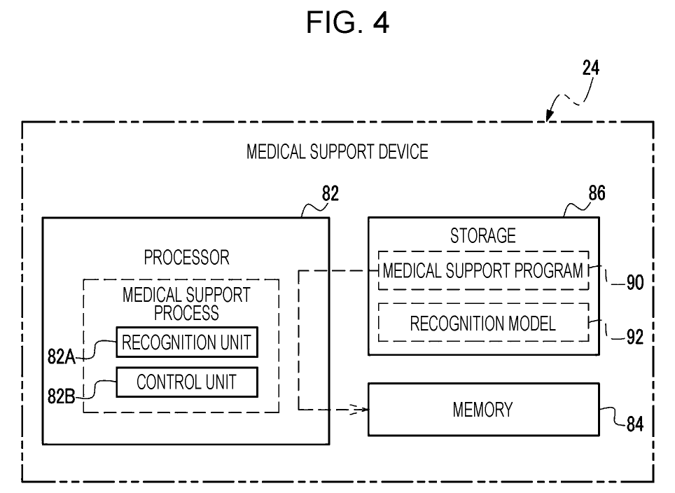

In view of such circumstances, in the present embodiment, as an example, as illustrated in FIG. 4, the processor 82 of the medical support device 24 performs a medical support process. FIG. 4 is a block diagram illustrating an example of main functions of the processor 82 included in the medical support device 24, and an example of information stored in the storage 86.

The storage 86 stores a medical support program 90. The medical support program 90 is an example of a "program" according to the present disclosure. The processor 82 reads the medical support program 90 from the storage 86 and executes the read medical support program 90 on the memory 84 to perform the medical support process. The medical support process is implemented by the processor 82 operating as a recognition unit 82A and a control unit 82B in accordance with the medical support program 90 executed on the memory 84.

The storage 86 stores a recognition model 92. As described in detail below, the recognition model 92 is used by the recognition unit 82A.

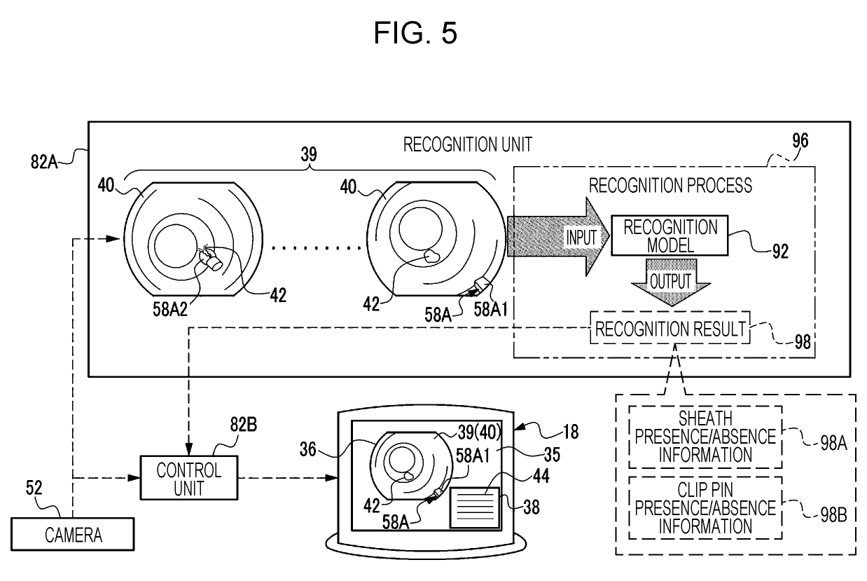

FIG. 5 is a conceptual diagram illustrating an example of the content of a process by the recognition unit 82A and the control unit 82B. As illustrated in FIG. 5, the recognition unit 82A and the control unit 82B acquire, from the camera 52, each of a plurality of frames 40 along a time series included in the endoscopic moving image 39 generated by imaging the inside of the colon 28 with the camera 52 in accordance with an imaging frame rate (e.g., several tens of frames/second), frame by frame along a time series. In the example illustrated in FIG. 5, the recognition unit 82A acquires a plurality of frames 40 showing a bleeding site 42 (e.g., an area where bleeding occurs as a result of excision of the lesion 41) and showing stages of the hemostasis treatment using the hemostat 58A. The frames 40 acquired from the camera 52 by the recognition unit 82A are acquired by the control unit 82B in synchronization with the recognition unit 82A. The stages of the hemostasis treatment using the hemostat 58A refer to, for example, a hemostasis preparation stage using the hemostat 58A and a hemostasis start stage using the hemostat 58A.

The hemostasis preparation stage is a stage where the bleeding site 42 is targeted (i.e., a stage in which the hemostasis treatment is prepared). At the stage where the bleeding site 42 is targeted, the frame 40 shows the sheath 58A1 (e.g., the sheath 58A1 in a state where the clip pin 58A2 does not protrude from the distal end thereof). Thus, the hemostasis preparation stage can be considered to be a stage at which the frame 40 shows the sheath 58A1 (e.g., the sheath 58A1 in a state where the clip pin 58A2 does not protrude from the distal end thereof).

The hemostasis start stage is a stage where a hemostasis treatment for the bleeding site 42 is started. At the stage where the hemostasis treatment for the bleeding site 42 is started, the frame 40 shows the clip pin 58A2 with which the bleeding site 42 is clipped and does not show the sheath 58A1. The hemostasis start stage can be considered to be a stage at which the frame 40 shows the clip pin 58A2 with which the bleeding site 42 is clipped and does not show the sheath 58A1. In the present embodiment, the stage of a change from a first state to a second state is employed as an example of the hemostasis start stage. The first state refers to a state in which the sheath 58A1 appears in the frame 40 obtained immediately before the latest frame 40. The second state refers to a state in which the latest frame 40 shows the clip pin 58A2 with which the bleeding site 42 is clipped and does not show the sheath 58A1.

In the present embodiment, a frame 40 in which the sheath 58A1 appears is an example of a "first frame" according to the present disclosure, and a frame 40 in which the clip pin 58A2 separated from the sheath 58A1 appears is an example of a "second frame" according to the present disclosure.

The control unit 82B outputs the endoscopic moving image 39 to the display device 18. For example, the control unit 82B displays the endoscopic moving image 39 in the first display region 36 as a live view image. That is, each time the control unit 82B acquires a frame 40 from the camera 52, the control unit 82B sequentially displays the acquired frame 40 in the first display region 36 in accordance with a display frame rate (e.g., several tens of frames/second). The control unit 82B further displays the medical information 44 in the second display region 38. Further, for example, the control unit 82B updates the content (e.g., the medical information 44) displayed in the second display region 38 in accordance with the content displayed in the first display region 36.

The recognition unit 82A uses the endoscopic moving image 39 acquired from the camera 52 to recognize the hemostat 58A appearing in the endoscopic moving image 39. That is, the recognition unit 82A sequentially performs a recognition process 96 on each of the plurality of frames 40 along a time series included in the endoscopic moving image 39 acquired from the camera 52 to recognize the hemostat 58A appearing in the frame 40. For example, the recognition unit 82A individually recognizes the sheath 58A1 and the clip pin 58A2. For example, the recognition of the clip pin 58A2 is recognition of the clip pin 58A2 in a state of being separated from the sheath 58A1.

Each time a frame 40 is acquired, the recognition unit 82A performs the recognition process 96 on the acquired frame 40. The recognition process 96 is a process for recognizing each of the sheath 58A1 and the clip pin 58A2 by a method using AI (i.e., an object recognition process using machine learning). In the present embodiment, for example, an AI-based object recognition process using a segmentation method (e.g., semantic segmentation, instance segmentation, and/or panoptic segmentation) is used as the recognition process 96.

A process using the recognition model 92 is performed as the recognition process 96. The recognition model 92 is a trained model for object recognition using an AI-based segmentation method. An example of the trained model for object recognition using an AI-based segmentation method is a model for semantic segmentation. An example of the model for semantic segmentation is an encoder-decoder structure model. An example of the encoder-decoder structure model is a U-Net model, an HRNet model, or the like. In the present embodiment, the recognition process 96 is an example of an "object recognition process" according to the present disclosure.

The recognition model 92 is optimized through machine learning using training data on a neural network. The training data is a dataset including a plurality of pieces of data (i.e., data for a plurality of frames) in which example data and ground-truth data are associated with each other.

The example data is various images assumed for the frames 40. The ground-truth data is ground-truth data (i.e., an annotation) for the example data. Here, as examples of the ground-truth data, annotations for identifying the sheaths of various hemostats appearing in images used as the example data, and annotations for identifying the clip pins of various hemostats appearing in images used as the example data are used.

The recognition unit 82A acquires a frame 40 from the camera 52 and inputs the acquired frame 40 to the recognition model 92. Accordingly, each time a frame 40 is input, the recognition model 92 recognizes whether the sheath 58A1 appears in the input frame 40 and recognizes whether the clip pin 58A2 appears in the input frame 40. Then, the recognition model 92 outputs a recognition result 98.

The recognition result 98 includes sheath presence/absence information 98A and clip pin presence/absence information 98B. The sheath presence/absence information 98A is information indicating whether the sheath 58A1 appears in the corresponding frame 40 (i.e., the frame 40 input to the recognition model 92). The clip pin presence/absence information 98B is information indicating whether the clip pin 58A2 appears in the corresponding frame 40 (i.e., the frame 40 input to the recognition model 92).

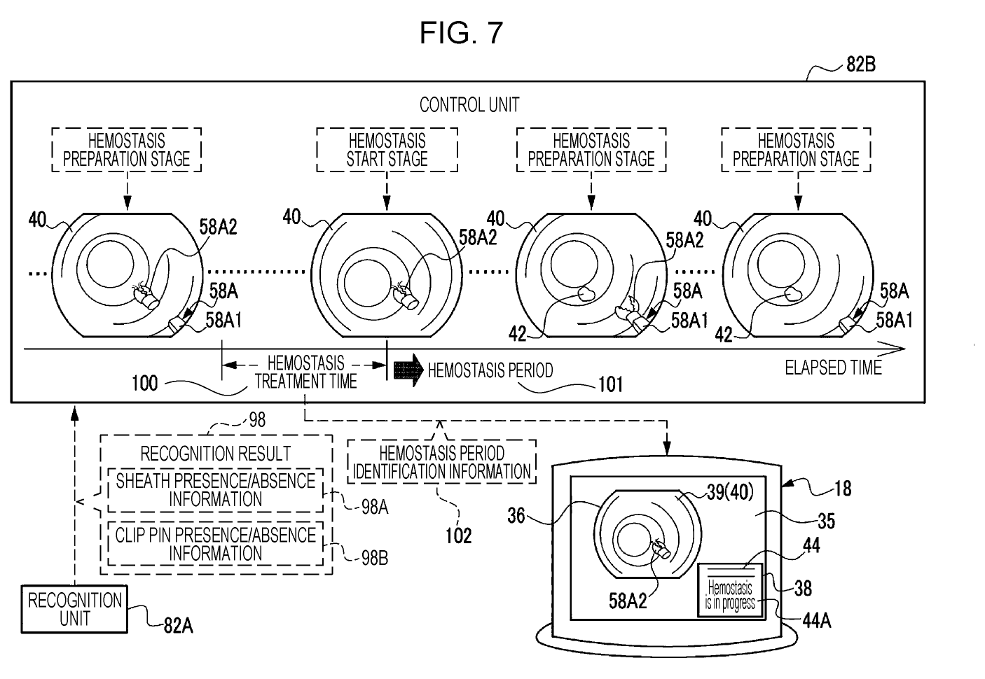

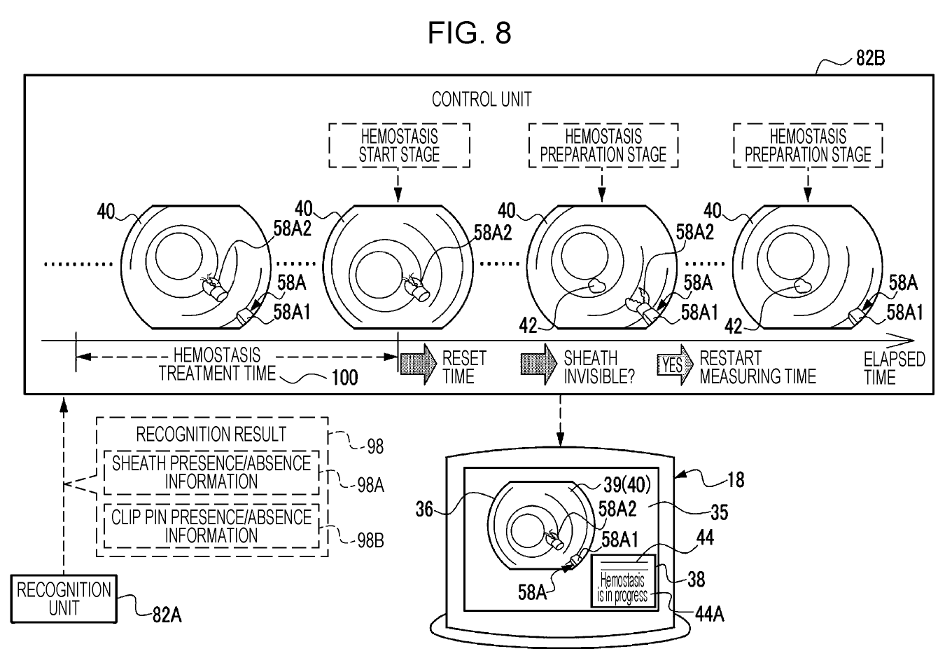

The recognition unit 82A acquires the recognition result 98 output from the recognition model 92 and outputs the acquired recognition result 98 to the control unit 82B. The control unit 82B executes a process using the recognition result 98 input from the recognition unit 82A (see, for example, FIGS. 7 and 8).

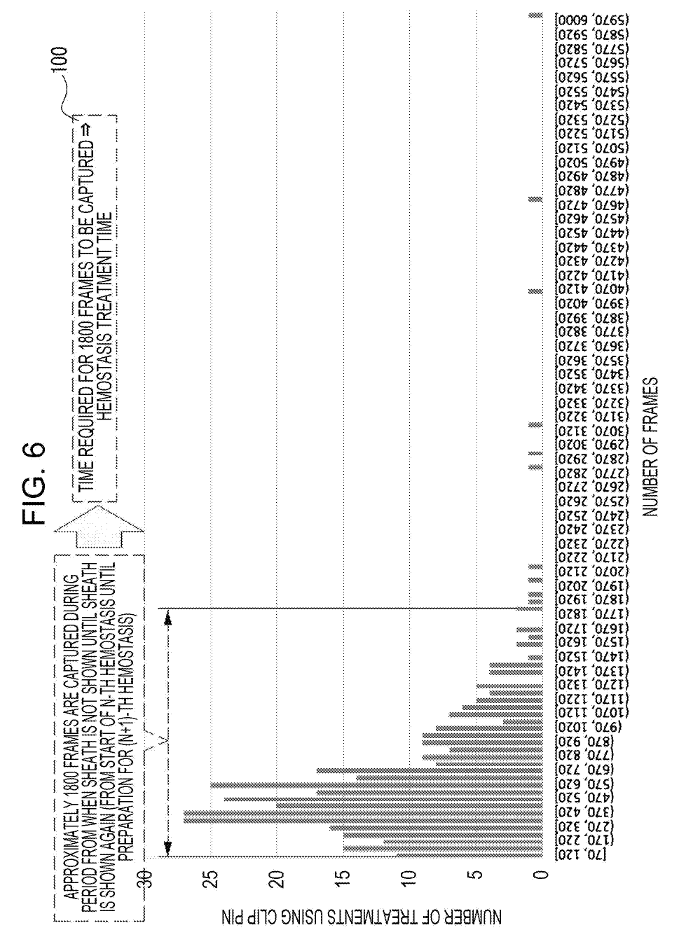

FIG. 6 is a graph (i.e., a histogram) illustrating an example of the relationship between the number of frames required for a single hemostasis treatment using the clip pin 58A2 to be completed in an endoscopic examination (i.e., the number of frames captured by the camera 52 during a period from the start to the end of a single hemostasis treatment using the clip pin 58A2) and the number of times a hemostasis treatment using the hemostat 58A is performed. The vertical axis illustrated in FIG. 6 represents the number of times the hemostasis treatment using the hemostat 58A is performed (i.e., the number of treatments by clip hemostasis using the clip pin 58A2), and the horizontal axis illustrated in FIG. 6 represents the number of frames. The graph illustrated in FIG. 6 is a graph generated from data obtained by performing an endoscopic examination a plurality of times on the colon 28 of a plurality of subjects 26.

As illustrated in FIG. 6, a hemostasis treatment time 100 is used in the medical support process according to the present embodiment. The hemostasis treatment time 100 is an example of a "certain period of time" according to the present disclosure. The hemostasis treatment time 100 is a certain period of time determined in advance as the time required for a single hemostasis treatment to be completed, and is statistically determined as the time required for the hemostasis treatment to be performed once.

In the example illustrated in FIG. 6, in most endoscopic examinations, a single hemostasis treatment is completed by the time approximately 1800 frames are captured by the camera 52 after one hemostasis treatment using the clip pin 58A2 is started.

In the example illustrated in FIG. 6, the timing at which one hemostasis treatment using the clip pin 58A2 is started is set to a timing at which the bleeding site 42 is clipped with the clip pin 58A2 and the sheath 58A1 exits the frame 40. In the example illustrated in FIG. 6, furthermore, the timing at which one hemostasis treatment using the clip pin 58A2 is completed is set to a timing at which approximately 1800 frames are captured by the camera 52 after one hemostasis treatment using the clip pin 58A2 is started.

The timing at which approximately 1800 frames are captured is the timing at which the sheath 58A1 enters the frame again in most endoscopic examinations (the timing at which the sheath 58A1 appears again in the frame 40). The state in which the sheath 58A1 enters the frame again means the state in which, after the completion of the N-th hemostasis treatment using the clip pin 58A2, preparation has begun for the (N+1)-th hemostasis treatment (e.g., a stage of searching for a location to be clipped with the clip pin 58A2 in the (N+1)-th endoscopic examination (i.e., the bleeding site 42 where the preceding hemostasis treatment was performed or a bleeding site 42 different from the bleeding site 42 where the preceding hemostasis treatment was performed)). The state in which preparation has begun for the (N+1)-th hemostasis treatment is the state in which the doctor 12 has determined that the N-th hemostasis treatment has been completed. Thus, the time required for approximately 1800 frames to be captured by the camera 52 after the N-th hemostasis treatment using the clip pin 58A2 has been started can be considered to be sufficient time from the start to the end of the N-th hemostasis treatment. In the example illustrated in FIG. 6, hemostasis treatments performed sporadically after approximately 1800 frames have been captured are highly likely to be irregular hemostasis treatments that rarely occur in normal endoscopic examinations, and can therefore be statistically regarded as noise.

In the medical support process according to the present embodiment, the time required for the camera 52 to capture 1800 frames is employed as the hemostasis treatment time 100. However, this is merely an example. For example, the hemostasis treatment time 100 may be the time required for the camera 52 to capture a number of frames corresponding to a statistical value of the number of times a hemostasis treatment is performed in the graph illustrated in FIG. 6, such as the time required for the camera 52 to capture a number of frames corresponding to the maximum value of the number of times a hemostasis treatment is performed in the graph illustrated in FIG. 6, the time required for the camera 52 to capture a number of frames corresponding to the mean of the number of times a hemostasis treatment is performed in the graph illustrated in FIG. 6, or the time required for the camera 52 to capture a number of frames corresponding to the median of the number of times a hemostasis treatment is performed in the graph illustrated in FIG. 6. While the time required for a number of frames corresponding to the statistical value to be captured is illustrated here, this is merely an example. The hemostasis treatment time 100 may be a time determined in advance for the statistical value.

Also, the time required for the camera 52 to capture a number of frames less than 1800 frames or the time required for the camera 52 to capture a number of frames greater than 1800 frames may be used as the hemostasis treatment time 100. The hemostasis treatment time 100 determined by default (e.g., the hemostasis treatment time 100 determined once in the manner described above) may be adjusted in accordance with an instruction given from the doctor 12 or others (e.g., an instruction received by the reception device 64 or the like). In any case, it is sufficient that the hemostasis treatment time 100 be a period of time defined such that a timing at which the bleeding site 42 is clipped with the clip pin 58A2 and the sheath 58A1 exits the frame 40 is defined as the start timing of a hemostasis treatment and a timing designated as a timing at which the hemostasis treatment is completed is defined as the end timing of the hemostasis treatment. The hemostasis treatment time 100 is preferably a period of time statistically derived in advance from the history of a plurality of hemostasis treatments that have been actually performed (e.g., data obtained by collecting the time required for each of the plurality of hemostasis treatments) or a period of time obtained by adjusting the period of time derived in this way.

The start timing of the hemostasis treatment need not be a timing at which the bleeding site 42 is clipped with the clip pin 58A2 and the sheath 58A1 exits the frame 40, and may be a timing at which a change occurs from a state where the bleeding site 42 is not clipped with the clip pin 58A2 to a state where the bleeding site 42 is clipped with the clip pin 58A2. In this case, for example, it is sufficient that the state where the bleeding site 42 is not clipped with the clip pin 58A2 and the state where the bleeding site 42 is clipped with the clip pin 58A2 be recognized by an AI-based object recognition process (e.g., an object recognition process using the recognition model 92) and/or a non-AI-based object recognition process (e.g., an object recognition process using template matching).

The start timing of the hemostasis treatment may be a timing at which a change occurs from a state where the frame 40 shows the sheath 58A1 to a state where the frame 40 does not show the sheath 58A1. In this case, for example, it is sufficient that the state where the frame 40 shows the sheath 58A1 and the state where the frame 40 does not show the sheath 58A1 be recognized by the AI-based object recognition process and/or the non-AI-based object recognition process.

The hemostasis treatment is a medical treatment performed during a period from when the stage changes from the hemostasis preparation stage to the hemostasis start stage until the hemostasis treatment time 100 elapses. In the present embodiment, the start timing of the hemostasis treatment is a timing at which the stage changes from the hemostasis preparation stage to the hemostasis start stage, and the end timing of the hemostasis treatment is a point in time at which the hemostasis treatment time 100 elapses after the change from the hemostasis preparation stage to the hemostasis start stage. That is, in the medical support process according to the present embodiment, the period from the start timing of the hemostasis treatment to the end timing of the hemostasis treatment is used as a hemostasis period during which the hemostasis treatment is performed.

FIG. 7 is a conceptual diagram illustrating an example of the content of a process in which the control unit 82B identifies the hemostasis treatment time 100. As illustrated in FIG. 7, the control unit 82B identifies the hemostasis preparation stage and the hemostasis start stage based on the recognition result 98 input from the recognition unit 82A. That is, the control unit 82B refers to the sheath presence/absence information 98A included in the recognition result 98 to determine whether the frame 40 shows the sheath 58A1. When the frame 40 shows the sheath 58A1, the control unit 82B determines that the stage where the frame 40 shows the sheath 58A1 is the hemostasis preparation stage.

Subsequently, the control unit 82B refers to the sheath presence/absence information 98A and the clip pin presence/absence information 98B included in the recognition result 98 to determine whether the frame 40 does not show the sheath 58A1 and shows the clip pin 58A2. When a frame 40 showing the clip pin 58A2 and not showing the sheath 58A1 is obtained after a frame 40 showing the sheath 58A1, the control unit 82B determines that the stage where the frame 40 showing the clip pin 58A2 and not showing the sheath 58A1 is obtained after the frame 40 showing the sheath 58A1 is the hemostasis start stage.

Here, the control unit 82B determines that the period from when the stage changes from the hemostasis preparation stage to the hemostasis start stage until the hemostasis treatment time 100 elapses is a hemostasis period 101. That is, the control unit 82B detects a change from the hemostasis preparation stage to the hemostasis start stage based on the recognition result 98 and starts measuring the hemostasis treatment time 100. The control unit 82B identifies, as the hemostasis period 101, a period from when the change from the hemostasis preparation stage to the hemostasis start stage is detected until the hemostasis treatment time 100 elapses. When the control unit 82B identifies the hemostasis period 101, the control unit 82B outputs hemostasis period identification information 102 that enables identification of the hemostasis period 101 to the display device 18.

An example of the hemostasis period identification information 102 is a text 44A that enables identification of the hemostasis period 101. An example of the text 44A is the text "Hemostasis is in progress". The content of the text 44A may be any content that allows the current time to be determined to be within the hemostasis period 101. While the text 44A is illustrated, an image that enables identification of the hemostasis period 101 may be used.

When the hemostasis period identification information 102 is output to the display device 18, the text 44A is displayed in the second display region 38 as a portion of the medical information 44 during the hemostasis period 101 (i.e., until the hemostasis treatment time 100 elapses). While an example is illustrated here in which the text 44A is visibly displayed, this is merely an example, and audio that can identify the hemostasis period 101 may be output from a speaker (not illustrated), or information that can identify the hemostasis period 101 may be printed on media (e.g., sheets) by a printer. Also, while an example is illustrated here in which the hemostasis period identification information 102 is output to the display device 18, the hemostasis period identification information 102 may be output to a server, a personal computer, a tablet terminal, and/or the like. The hemostasis period identification information 102 may be recorded in an electronic medium such as an electronic medical record.

FIG. 8 is a conceptual diagram illustrating an example of the content of a process performed by the control unit 82B in a case where the sheath 58A1 appears in the frame 40 before the hemostasis treatment time 100 elapses after the change from the hemostasis preparation stage to the hemostasis start stage. As illustrated in FIG. 8, in some cases, the sheath 58A1 may appear in the frame 40 before the hemostasis treatment time 100 elapses after the change from the hemostasis preparation stage to the hemostasis start stage. For example, body motion, hand motion by the doctor 12, and/or the like may cause the sheath 58A1 to appear in the frame 40 at a timing unintended by the doctor 12 (i.e., a timing at which the hemostasis treatment is not completed). Also, for example, although the hemostasis treatment has not been completed, the doctor 12 may attempt to perform the next hemostasis treatment, and the sheath 58A1 may appear in the frame 40.

In this manner, in a case where the sheath 58A1 appears in the frame 40 before the hemostasis treatment time 100 elapses after a change from the hemostasis preparation stage to the hemostasis start stage, it is preferable that the doctor 12 grasp that the current time is within the hemostasis period 101.

Accordingly, the control unit 82B refers to the recognition result 98 to determine whether a frame 40 in which the sheath 58A1 appears has been obtained by the recognition unit 82A before the hemostasis treatment time 100 elapses after a change from the hemostasis preparation stage to the hemostasis start stage. If a frame 40 in which the sheath 58A1 appears is obtained by the recognition unit 82A (i.e., if the recognition unit 82A recognizes that the sheath 58A1 is in frame), the control unit 82B resets the hemostasis treatment time 100. Subsequently, if a frame 40 in which the sheath 58A1 does not appear is obtained by the recognition unit 82A (i.e., if the recognition unit 82A recognizes that the sheath 58A1 is out of frame), the control unit 82B restarts measuring the hemostasis treatment time 100 to start the hemostasis period 101 again.

In the present embodiment, a frame 40 in which the sheath 58A1 appears before the hemostasis treatment time 100 elapses after a change from the hemostasis preparation stage to the hemostasis start stage is an example of a "third frame" according to the present disclosure.

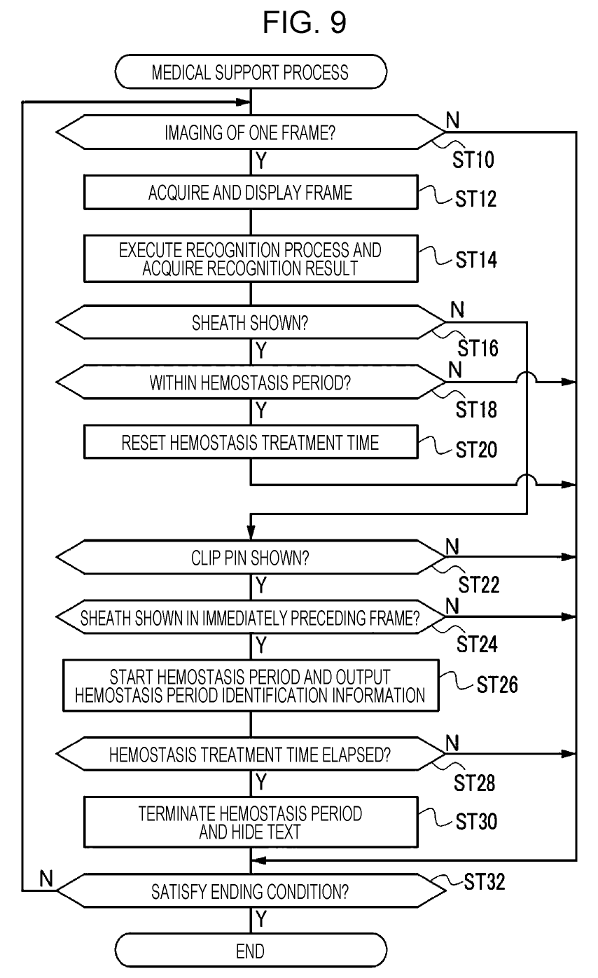

Next, the operation of a portion of the endoscope apparatus 10 according to the present disclosure will be described with reference to FIG. 9. The flow of the medical support process illustrated in FIG. 9 is an example of a "medical support method" according to the present disclosure. For convenience of description, a description will be given of a case where the medical support process is performed by the processor 82 in a case where the bleeding site 42 is generated by excision of the lesion 41 from the intestinal wall 32 in an endoscopic examination.

In the medical support process illustrated in FIG. 9, first, in step ST10, the recognition unit 82A determines whether imaging of one frame has been performed in the colon 28 by the camera 52. If imaging of one frame has not been performed in the colon 28 by the camera 52 in step ST10, the determination is negative, and the medical support process proceeds to step ST32. If imaging of one frame has been performed in the colon 28 by the camera 52 in step ST10, the determination is positive, and the medical support process proceeds to step ST12.

In step ST12, the recognition unit 82A and the control unit 82B acquire a frame 40 obtained by imaging the colon 28 with the camera 52. Then, the control unit 82B displays the frame 40 in the first display region 36 (see FIGS. 5, 7 and 8). After the processing of step ST12 is performed, the medical support process proceeds to step ST14.

In step ST14, the recognition unit 82A performs the recognition process 96 on the frame 40 acquired in step ST12. The control unit 82B acquires the recognition result 98, which is obtained by the recognition unit 82A performing the recognition process 96. After the processing of step ST14 is performed, the medical support process proceeds to step ST16.

In step ST16, the control unit 82B refers to the recognition result 98 acquired in step ST14 and determines whether the frame 40 acquired in step ST12 shows the sheath 58A1. If the frame 40 acquired in step ST12 does not show the sheath 58A1 in step ST16, the determination is negative, and the medical support process proceeds to step ST22. If the frame 40 acquired in step ST12 shows the sheath 58A1 in step ST16, the determination is positive, and the medical support process proceeds to step ST18.

In step ST18, the control unit 82B determines whether the endoscopic examination enters the hemostasis period 101. If the endoscopic examination does not enter the hemostasis period 101 in step ST18 (i.e., if the current time is not within the hemostasis period 101), the determination is negative, and the medical support process proceeds to step ST32. If the endoscopic examination enters the hemostasis period 101 in step ST18, the determination is positive, and the medical support process proceeds to step ST20.

In step ST20, the control unit 82B resets the hemostasis treatment time 100. After the processing of step ST20 is performed, the medical support process proceeds to step ST32.

In step ST22, the control unit 82B refers to the recognition result 98 acquired in step ST14 to determine whether the frame 40 acquired in step ST12 shows the clip pin 58A2. If the frame 40 acquired in step ST12 does not show the clip pin 58A2 in step ST22, the determination is negative, and the medical support process proceeds to step ST32. If the frame 40 acquired in step ST12 shows the clip pin 58A2 in step ST22, the determination is positive, and the medical support process proceeds to step ST24.

In step ST24, the control unit 82B determines whether the sheath 58A1 is shown in the frame 40 acquired in step ST12 immediately before the frame 40 used for the determination in step ST22. If the frame 40 acquired in step ST12 immediately before the frame 40 used for the determination in step ST22 does not show the sheath 58A1 in step ST24, the determination is negative, and the medical support process proceeds to step ST32. If the frame 40 acquired in step ST12 immediately before the frame 40 used for the determination in step ST22 shows the sheath 58A1 in step ST24, the determination is positive, and the medical support process proceeds to step ST26.

In step ST26, the control unit 82B starts the hemostasis period 101. The start of the hemostasis period 101 is implemented by the control unit 82B starting measuring the hemostasis treatment time 100. Then, the control unit 82B outputs the hemostasis period identification information 102 to the display device 18. As a result, the text 44A is displayed in the second display region 38 (see FIG. 7). After the processing of step ST26 is performed, the medical support process proceeds to step ST28.

In step ST28, the control unit 82B determines whether the hemostasis treatment time 100 has elapsed since the measurement of the hemostasis treatment time 100 was started. If the hemostasis treatment time 100 has not elapsed in step ST28 since the measurement of the hemostasis treatment time 100 was started, the determination is negative, and the medical support process proceeds to step ST32. If the hemostasis treatment time 100 has elapsed in step ST28 since the measurement of the hemostasis treatment time 100 was started, the determination is positive, and the medical support process proceeds to step ST30.

In step ST30, the control unit 82B terminates the hemostasis period 101. The termination of the hemostasis period 101 is implemented by the control unit 82B terminating the measurement of the hemostasis treatment time 100. Then, the control unit 82B hides the text 44A in the second display region 38. That is, the text 44A is deleted from the second display region 38. After the processing of step ST30 is performed, the medical support process proceeds to step ST32.

In step ST32, the control unit 82B determines whether a condition for ending the medical support process is satisfied. An example of the condition for ending the medical support process is a condition in which an instruction to end the medical support process is provided to the endoscope apparatus 10 (e.g., a condition in which the instruction to end the medical support process is received by the reception device 64).

If the condition for ending the medical support process is not satisfied in step ST32, the determination is negative, and the medical support process proceeds to step ST10. If the condition for ending the medical support process is satisfied in step ST32, the determination is positive, and the medical support process ends.

As described above, in the endoscope apparatus 10, the recognition unit 82A acquires a plurality of frames 40 that are obtained in time series by imaging the inside of the colon 28 with the camera 52 and that show stages of the hemostasis treatment. Then, the control unit 82B outputs the hemostasis period identification information 102, which enables identification of the hemostasis period 101 from which the stages of the hemostasis treatment shown in the plurality of frames 40 acquired by the recognition unit 82A change from the hemostasis preparation stage to the hemostasis start stage until the hemostasis treatment time 100 elapses, to the display device 18. The screen 35 of the display device 18 (as an example, the second display region 38) displays the text 44A, which enables identification of the hemostasis period 101. This allows the doctor 12 to grasp that the current time is in the hemostasis period 101 (i.e., the period during which a hemostasis treatment is performed with the hemostat 58A).

Further, a time statistically determined as the time required for a hemostasis treatment using the hemostat 58A to be performed once (as an example, a time determined based on the graph illustrated in FIG. 6) is used as the hemostasis treatment time 100 here. This can make it difficult for the (N+1)-th hemostasis treatment to start before, for example, the N-th hemostasis treatment is completed. Additionally, since the doctor 12 can accurately grasp the end timing of one hemostasis treatment, the number of times the hemostasis treatment has been performed can also be accurately counted.

In the endoscope apparatus 10, furthermore, the control unit 82B detects a change from the hemostasis preparation stage to the hemostasis start stage, based on the recognition result 98 obtained by the recognition unit 82A performing the recognition process 96 using the recognition model 92 on the plurality of frames 40. This enables detection of a change from the hemostasis preparation stage to the hemostasis start stage without imposing a burden on the doctor 12, as compared to a case where the doctor 12 determines a change from the hemostasis preparation stage to the hemostasis start stage by observing a plurality of frames 40.

In this manner, a period from when the change from the hemostasis preparation stage to the hemostasis start stage is detected until the hemostasis treatment time 100 elapses is set as the hemostasis period 101. The hemostasis period 101 is notified to the doctor 12 through the text 44A displayed in the second display region 38. In this manner, a period from when a change from the hemostasis preparation stage to the hemostasis start stage is detected until the hemostasis treatment time 100 elapses is set as the hemostasis period 101 and is notified to the doctor 12. This allows the doctor 12 to grasp the hemostasis period 101 without imposing a burden on the doctor 12.

In the endoscope apparatus 10, furthermore, a stage where a frame 40 showing the sheath 58A1 is obtained is set as the hemostasis preparation stage, and a stage where a frame 40 not showing the sheath 58A1 is obtained after the frame 40 showing the sheath 58A1 is set as the hemostasis start stage. Accordingly, the timing at which the hemostasis treatment starts, that is, the start timing of the hemostasis period 101, can be determined in a simple manner. As a result, the hemostasis period 101 can also be determined in a simple manner.

In the endoscope apparatus 10, furthermore, a stage where a frame 40 showing the sheath 58A1 is obtained is set as the hemostasis preparation stage, and a stage where a frame 40 showing the clip pin 58A2, which is separated from the sheath 58A1, is obtained after the frame 40 showing the sheath 58A1 is set as the hemostasis start stage. Accordingly, the timing at which the hemostasis treatment starts, that is, the start timing of the hemostasis period 101, can be determined in a simple manner. As a result, the hemostasis period 101 can also be determined in a simple manner. While a frame 40 showing the clip pin 58A2, which is separated from the sheath 58A1, is illustrated here, this is merely an example, and the present disclosure is also applicable to a frame 40 showing the clip pin 58A2 remaining unseparated from the sheath 58A1.

In the endoscope apparatus 10, furthermore, a stage where a frame 40 showing the sheath 58A1 is obtained is set as the hemostasis preparation stage, and a stage where a frame 40 showing the clip pin 58A2, which is separated from the sheath 58A1, and not showing the sheath 58A1 is obtained after the frame 40 showing the sheath 58A1 is set as the hemostasis start stage. Accordingly, the timing at which the hemostasis treatment starts, that is, the start timing of the hemostasis period 101, can be determined in a simple manner. As a result, the hemostasis period 101 can also be determined in a simple manner.

In the endoscope apparatus 10, furthermore, the hemostasis treatment time 100 is reset in a case where a frame 40 showing the sheath 58A1 is obtained while the hemostasis treatment time 100 is being measured (i.e., during the hemostasis period 101). That is, under the condition in which the hemostasis treatment time 100 is determined in advance as the time required for a single hemostasis treatment to be completed, if a frame 40 showing the sheath 58A1 is obtained before the hemostasis treatment time 100 elapses after a change from the hemostasis preparation stage to the hemostasis start stage, the hemostasis treatment time 100 is reset. This can prevent the doctor 12 from starting the next hemostasis treatment before the hemostasis treatment is completed, or prevent the doctor 12 or others from erroneously recognizing that a hemostasis treatment has been performed once although the hemostasis treatment has not been completed.

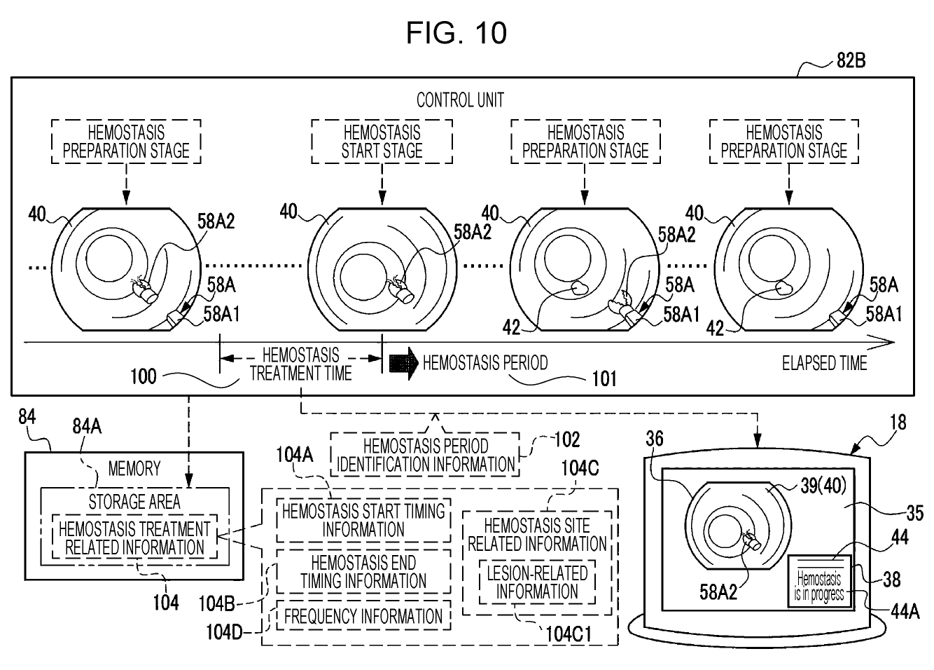

While the embodiment described above illustrates an example in which the control unit 82B outputs the hemostasis period identification information 102 to the display device 18 during the hemostasis period 101, the present disclosure is not limited to this example. For example, as illustrated in FIG. 10, the control unit 82B may store hemostasis treatment related information 104 regarding a hemostasis treatment performed during the hemostasis period 101 in a storage area 84A of the memory 84, on condition that the hemostasis period 101 has started. This makes it easy to perform processing using the hemostasis treatment related information 104.

The hemostasis treatment related information 104 may include the hemostasis period identification information 102 or may include a portion of the hemostasis period identification information 102. This makes it easy to perform processing using the hemostasis period identification information 102 or the portion of the hemostasis period identification information 102.

An example of information included in the portion of the hemostasis period identification information 102 included in the hemostasis treatment related information 104 is hemostasis start timing information 104A that enables identification of the start timing of the hemostasis period 101 (e.g., information indicating the start time of the hemostasis period 101, and/or hemostasis end timing information 104B that enables identification of the end timing of the hemostasis period 101 (e.g., information indicating the end time of the hemostasis period 101). The hemostasis start timing information 104A is an example of "start timing information" according to the present disclosure, and the hemostasis end timing information 104B is an example of "end timing information" according to the present disclosure.

If the hemostasis start timing information 104A is stored in the storage area 84A, the doctor 12 can grasp the start timing of the hemostasis period 101 from the hemostasis start timing information 104A stored in the storage area 84A. If the hemostasis end timing information 104B is stored in the storage area 84A, the doctor 12 can grasp the end timing of the hemostasis period 101 from the hemostasis end timing information 104B stored in the storage area 84A.

The hemostasis treatment related information 104 may also include hemostasis site related information 104C. The hemostasis site related information 104C is an example of "hemostasis site related information" according to this disclosure. The hemostasis site related information 104C refers to information related to a site where a hemostasis treatment has been performed in the colon 28. If the hemostasis site related information 104C is stored in the storage area 84A, the doctor 12 or others can refer to the hemostasis site related information 104C stored in the storage area 84A to determine what medical treatment is necessary for the site where the hemostasis treatment has been performed in the colon 28.

An example of information included in the hemostasis site related information 104C is lesion-related information 104C1. The lesion-related information 104C1 is an example of "lesion-related information" according to the present disclosure. The lesion-related information 104C1 refers to information related to the lesion 41 present at the site where the hemostasis treatment has been performed. In this manner, if the hemostasis site related information 104C includes the lesion-related information 104C1, the doctor 12 or others can refer to the lesion-related information 104C1 stored in the storage area 84A to determine what medical treatment is necessary for the lesion 41 present in the site where hemostasis has been performed, what medical treatment is necessary for the site where the lesion 41 was present (e.g., the bleeding site 42), or what medical treatment is necessary for the site where hemostasis is completed (e.g., the site clipped with the clip pin 58A2).

The hemostasis treatment related information 104 may also include frequency information 104D. The frequency information 104D is an example of "frequency information" according to the present disclosure. The frequency information 104D is information that enables identification of the number of times a hemostasis treatment has been performed in the colon 28 (e.g., the number of hemostasis treatments performed on the bleeding site 42 for one lesion 41 or the number of clip pins 58A2 remaining placed in the colon 28). In this manner, if the hemostasis treatment related information 104 includes the frequency information 104D, the doctor 12 or others can refer to the frequency information 104D stored in the storage area 84A to easily grasp the number of times the hemostasis treatment has been performed in the colon 28. As a result, it is possible to determine a necessary medical treatment in accordance with the number of times the hemostasis treatment has been performed in the colon 28.

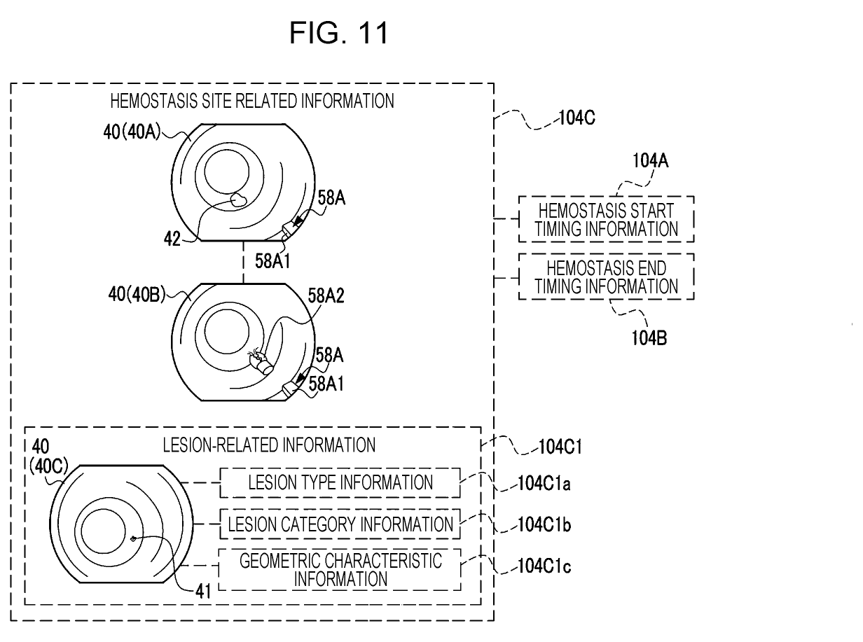

As an example, as illustrated in FIG. 11, the hemostasis site related information 104C may include a pre-hemostasis image 40A. The pre-hemostasis image 40A is an example of a "pre-hemostasis image" according to the present disclosure. The pre-hemostasis image 40A is a frame 40 obtained by imaging, with the camera 52, a pre-hemostasis condition of the bleeding site 42 where the hemostasis treatment has been performed. If the hemostasis site related information 104C includes the pre-hemostasis image 40A, the doctor 12 or others can refer to the pre-hemostasis image 40A stored in the storage area 84A to grasp the condition of the bleeding site 42 before hemostasis.

The hemostasis site related information 104C may also include a post-hemostasis image 40B. The post-hemostasis image 40B is an example of a "post-hemostasis image" according to the present disclosure. The post-hemostasis image 40B is a frame 40 obtained by imaging, with the camera 52, the site where the hemostasis treatment has been performed. If the hemostasis site related information 104C includes the post-hemostasis image 40B, the doctor 12 or others can refer to the post-hemostasis image 40B stored in the storage area 84A to grasp the condition of the site where the hemostasis treatment has been performed.

The lesion-related information 104C1 may include a lesion image 40C. The lesion image 40C is an example of a "lesion image" according to the present disclosure. The lesion image 40C is a frame 40 obtained by imaging the lesion 41 with the camera 52. If the lesion-related information 104C1 includes the lesion image 40C, the doctor 12 or others can refer to the frame 40 included in the lesion image 40C stored in the storage area 84A to determine what medical treatment is necessary for the lesion 41 present in the site where hemostasis has been performed, what medical treatment is necessary for the site where the lesion 41 was present (e.g., the bleeding site 42), or what medical treatment is necessary for the site where hemostasis is completed (e.g., the site clipped with the clip pin 58A2).

The lesion-related information 104C1 may include lesion type information 104C1a and/or lesion category information 104C1b. The lesion type information 104C1a is information indicating the type of the lesion 41. Examples of the type of the lesion 41 include neoplastic polyps and non-neoplastic polyps. The types of the neoplastic polyps include, for example, adenomatous polyps (e.g., SSL). The types of the non-neoplastic polyps include, for example, hamartoma polyps, hyperplastic polyps, and inflammatory polyps. The types exemplified here are types expected in advance as possible types of the lesion 41 when the endoscopic examination is performed on the colon 28, and the type of the lesion 41 differs depending on the organ on which the endoscopic examination is performed.

The lesion category information 104C1b is information indicating the category of the lesion 41. Examples of the category of the lesion 41 include pedunculated, subpedunculated, sessile, surface elevated, surface flat, and surface depressed types.

The lesion-related information 104C1 may include geometric characteristic information 104C1c. The geometric characteristic information 104C1c is information indicating the geometric characteristics of the lesion 41. An example of the geometric characteristics of the lesion 41 is information (e.g., coordinates) that enables identification of the position, shape, and/or the like of an image region indicating the lesion 41 in the lesion image 40C.

For example, the lesion type information 104C1a, the lesion category information 104C1b, and/or the geometric characteristic information 104C1c are information obtained by performing an object recognition process (e.g., an object recognition process using AI and/or an object recognition process using template matching) on the frame 40. The lesion type information 104C1a, the lesion category information 104C1b, and/or the geometric characteristic information 104C1c may be received by the reception device 64. In this case, the control unit 82B causes the lesion type information 104C1a, the lesion category information 104C1b, and/or the geometric characteristic information 104C1c, which are received by the reception device 64, to be included in the lesion-related information 104C1.

The lesion type information 104C1a, the lesion category information 104C1b, and/or the geometric characteristic information 104C1c may be associated with the lesion image 40C. With this configuration, the control unit 82B displays the lesion image 40C on the screen 35, and also displays the lesion type information 104C1a, the lesion category information 104C1b, and/or the geometric characteristic information 104C1c associated with the lesion image 40C. This allows the doctor 12 to observe the lesion image 40C through the screen 35 and grasp the type of the lesion 41, the category of the lesion 41, and/or the geometric characteristics of the lesion 41 appearing in the lesion image 40C.

The hemostasis start timing information 104A and/or the hemostasis end timing information 104B may be associated with the hemostasis site related information 104C related to the hemostasis start timing information 104A and/or the hemostasis end timing information 104B (e.g., the hemostasis site related information 104C obtained during the hemostasis period 101 identified by the hemostasis start timing information 104A and/or the hemostasis end timing information 104B). This allows the doctor 12 to grasp the hemostasis site related information 104C through the screen 35 or the like and also grasp the hemostasis start timing information 104A and/or the hemostasis end timing information 104B associated with the hemostasis site related information 104C through the screen 35 or the like.

In the example illustrated in FIG. 10, an example is illustrated in which the storage area 84A stores the hemostasis treatment related information 104; this is merely an example. At least part of the information included in the hemostasis treatment related information 104 may be stored in the storage 76 and/or 86 or the like. Alternatively, at least part of the information included in the hemostasis treatment related information 104 may be stored in an electronic medical record. Alternatively, at least part of the information included in the hemostasis treatment related information 104 may be stored in a server, a personal computer, and/or a tablet terminal. At least part of the information included in the hemostasis treatment related information 104 may be printed on media by a printer. Alternatively, at least part of the information included in the hemostasis treatment related information 104 may be output as audio from a speaker.

Alternatively, at least part of the information included in the hemostasis treatment related information 104 may be used as input information for so-called generative AI. Information output from the generative AI may be stored in the various storage areas described above (e.g., the storage 76 and/or 86), may be displayed as the medical information 44 on the display device 18, may be printed on media by a printer, or may be output as audio from a speaker. An example of the generative AI is ChatGPT using GPT-4 (retrieved from the Internet: https://openai.com/gpt-4), GPT-4-32k, or the like. When at least part of the information included in the hemostasis treatment related information 104 is used for ChatGPT, for example, the at least part of the information included in the hemostasis treatment related information 104 is used for instruction data (e.g., a prompt) to be input to ChatGPT. An example of information output from ChatGPT is information related to a medical treatment derived from the hemostasis treatment related information 104 input to ChatGPT (e.g., information indicating the content of a medical treatment recommended to be performed after the hemostasis treatment).

While the embodiment described above illustrates the AI-based recognition process 96 using the segmentation method, the present disclosure is not limited to this. For example, the present disclosure is also applicable to AI-based recognition processes using a bounding box method.

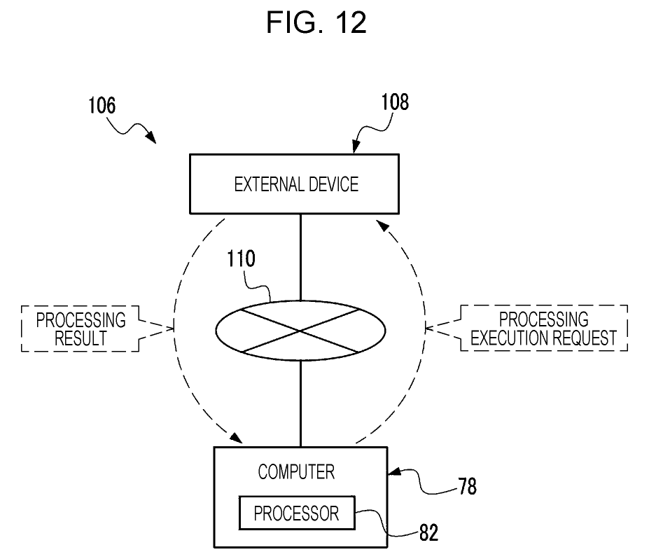

While the embodiment described above illustrates an example in which the medical support process is performed by the computer 78, the present disclosure is not limited to this example, and at least some of the processes included in the medical support process may be performed by a device provided external to the computer 78. An example of this case will be described hereinafter with reference to FIG. 12.

FIG. 12 is a conceptual diagram illustrating an example configuration of an endoscope apparatus 106. The endoscope apparatus 106 is an example of the "endoscope apparatus" according to the present disclosure. Unlike the endoscope apparatus 10 described in the embodiment described above, the endoscope apparatus 106 has an external device 108.

The external device 108 is communicably connected to the computer 78 via a network 110 (e.g., a WAN, a LAN, and/or the like).

An example of the external device 108 is at least one server that transmits and receives data to and from the computer 78 directly or indirectly via the network 110. The external device 108 receives a processing execution request provided from the processor 82 of the computer 78 via the network 110. Then, the external device 108 executes processing in accordance with the received processing execution request, and transmits a processing result to the computer 78 via the network 110. In the computer 78, the processor 82 receives the processing result transmitted from the external device 108 via the network 110 and executes processing using the received processing result.

Examples of the processing execution request include an instruction to cause the external device 108 to execute at least a portion of the medical support process. A first example of at least a portion of the medical support process (i.e., a process to be executed by the external device 108) is the recognition process 96. In this case, the external device 108 executes the recognition process 96 in accordance with a processing execution request provided from the processor 82 via the network 110, and transmits the recognition result 98 to the computer 78 via the network 110. In the computer 78, the processor 82 receives the recognition result 98 and executes processing similar to that in the embodiment described above by using the received recognition result 98.

A second example of at least a portion of the medical support process (i.e., a process to be executed by the external device 108) is a process performed by the control unit 82B. In this case, the external device 108 executes the process performed by the control unit 82B in accordance with a processing execution instruction provided from the processor 82 via the network 110, and transmits a processing result (e.g., the hemostasis period identification information 102, the hemostasis treatment related information 104, and/or the like) to the computer 78 via the network 110. In the computer 78, the processor 82 receives the processing result and executes processing similar to that in the embodiment described above (e.g., display using the display device 18, storage in the storage area 84A, and/or the like) by using the received processing result.

For example, the external device 108 is implemented by cloud computing. The cloud computing is merely an example, and the external device 108 may be implemented by network computing such as fog computing, edge computing, or grid computing. Instead of the server, at least one personal computer or the like may be used as the external device 108. Alternatively, the external device 108 may be an arithmetic device with a communication function mounted with a plurality of types of AI functions.

While the embodiment described above illustrates an example in which the medical support program 90 is stored in the storage 86, the present disclosure is not limited to this example. For example, the medical support program 90 may be stored in a portable non-transitory computer-readable storage medium such as an SSD or a USB memory. The medical support program 90 stored in the non-transitory storage medium is installed in the computer 78 of the endoscope apparatus 10. The processor 82 executes the medical support process in accordance with the medical support program 90.

Alternatively, the medical support program 90 may be stored in a storage device of another computer, a server, or the like connected to the endoscope apparatus 10 via a network, and the medical support program 90 may be downloaded in response to a request from the endoscope apparatus 10 and installed in the computer 78.

It is not necessary to store the entire medical support program 90 in a storage device of another computer, a server device, or the like connected to the endoscope apparatus 10 or to store the entire medical support program 90 in the storage 86, and a portion of the medical support program 90 may be stored.

Examples of a hardware resource that executes the medical support process may include the following various processors. The processors include, for example, a CPU that is a general-purpose processor configured to execute software, that is, a program, to function as a hardware resource that executes the medical support process. The processors further include, for example, a dedicated electric circuit that is a processor having a circuit configuration designed specifically for executing specific processing, such as an FPGA, a PLD, or an ASIC. Each of the processors incorporates or is connected to memory, and uses the memory to execute the medical support process.

The hardware resource that executes the medical support process may be configured as one of the various processors or as a combination of two or more processors of the same type or different types (e.g., a combination of a plurality of FPGAs or a combination of a CPU and an FPGA). The hardware resource that executes the medical support process may be a single processor.

Examples of configuring the hardware resource as a single processor include, first, a form in which a single processor is configured as a combination of one or more CPUs and software and the processor functions as a hardware resource that executes the medical support process. The examples include, second, a form in which, as typified by an SoC or the like, a processor is used in which the functions of the entire system including a plurality of hardware resources that execute the medical support process are implemented as one IC chip. As described above, the medical support process is implemented by using one or more of the various processors described above as hardware resources.

More specifically, the hardware structure of these various processors may be an electric circuit in which circuit elements such as semiconductor elements are combined. The medical support process described above is merely an example. Thus, it goes without saying that unnecessary steps may be deleted, new steps may be added, or the processing order may be changed without departing from the gist.

The contents described and illustrated above are detailed descriptions of portions related to the present disclosure and are merely examples of the present disclosure. For example, the description related to the configurations, functions, operations, and effects described above is a description related to examples of the configurations, functions, operations, and effects of portions related to the present disclosure. Thus, it goes without saying that unnecessary portions may be deleted from the contents described and illustrated above or new elements may be added to or substituted for the contents described and illustrated above without departing from the gist of the present disclosure. In addition, to avoid complexity and facilitate understanding of portions related to the present disclosure, description of common general technical knowledge and the like that do not require specific description to implement the present disclosure is omitted in the contents described and illustrated above.

All publications, patent applications, and technical standards described herein are incorporated herein by reference to the same extent as if each individual publication, patent application, and technical standard were specifically and individually indicated to be incorporated by reference.

What is claimed is:

1. A medical support device comprising a processor,
the processor is configured to:
acquire a plurality of frames obtained in time series by imaging, with a camera, an inside of a luminal organ into which a hemostat is inserted, the plurality of frames showing stages of a hemostasis treatment using the hemostat; and
output hemostasis period identification information that enables determination that a period from when the stages shown in the plurality of acquired frames change from a hemostasis preparation stage using the hemostat to a hemostasis start stage using the hemostat until a certain period of time elapses is a hemostasis period during which the hemostasis treatment is performed.

2. The medical support device according to claim 1, wherein
the processor is configured to perform an object recognition process based on the plurality of acquired frames to detect a change from the hemostasis preparation stage to the hemostasis start stage, and
the hemostasis period is a period from when the change is detected by the processor until the certain period of time elapses.

3. The medical support device according to claim 2, wherein
the object recognition process is implemented by using a model obtained by machine learning performed on a neural network.

4. The medical support device according to claim 1, wherein
the hemostat has a sheath,
the plurality of frames include a first frame and a second frame,
the first frame is a frame that shows the sheath,
the second frame is a frame that does not show the sheath,
the hemostasis preparation stage is a stage in which the first frame is obtained, and
the hemostasis start stage is a stage in which the second frame is obtained subsequent to the first frame.

5. The medical support device according to claim 1, wherein
the hemostat has a sheath and a clip pin,
the plurality of frames include a first frame and a second frame,
the first frame is a frame that shows the sheath,
the second frame is a frame that shows the clip pin,
the hemostasis preparation stage is a stage in which the first frame is obtained, and
the hemostasis start stage is a stage in which the second frame is obtained subsequent to the first frame.

6. The medical support device according to claim 1, wherein
the hemostat has a sheath and a clip pin,
the plurality of frames include a first frame and a second frame,
the first frame is a frame that shows the sheath,
the second frame is a frame that shows the clip pin and does not show the sheath,
the hemostasis preparation stage is a stage in which the first frame is obtained, and
the hemostasis start stage is a stage in which the second frame is obtained subsequent to the first frame.

7. The medical support device according to claim 4, wherein
the plurality of frames further include a third frame,
the third frame is a frame that shows the sheath, and
the certain period of time is reset in a case where the third frame is obtained before the certain period of time elapses after a change from the hemostasis preparation stage to the hemostasis start stage.

8. The medical support device according to claim 1, wherein
hemostasis treatment related information regarding the hemostasis treatment within the hemostasis period is stored in a storage area.

9. The medical support device according to claim 8, wherein
the hemostasis treatment related information includes the hemostasis period identification information or a portion of the hemostasis period identification information.

10. The medical support device according to claim 9, wherein
the portion includes start timing information that enables identification of a start timing of the hemostasis period and/or end timing information that enables identification of an end timing of the hemostasis period.

11. The medical support device according to claim 8, wherein
the hemostasis treatment related information includes hemostasis site related information regarding a site where the hemostasis treatment has been performed in the luminal organ.

12. The medical support device according to claim 11, wherein
the hemostasis site related information includes lesion-related information regarding a lesion present in the site where the hemostasis treatment has been performed.

13. The medical support device according to claim 12, wherein
the lesion-related information includes a lesion image obtained by imaging the lesion with the camera.

14. The medical support device according to claim 11, wherein
the hemostasis site related information includes a post-hemostasis image obtained by imaging, with the camera, the site where the hemostasis treatment has been performed.

15. The medical support device according to claim 11, wherein
the hemostasis site related information includes a pre-hemostasis image obtained by imaging, with the camera, a pre-hemostasis condition of the site where the hemostasis treatment has been performed.

16. The medical support device according to claim 8, wherein
the hemostasis treatment related information includes frequency information that enables identification of a number of times the hemostasis treatment has been performed in the luminal organ.

17. The medical support device according to claim 1, wherein
the certain period of time is a period of time statistically determined as a time required for the hemostasis treatment to be performed once using the hemostat.

18. An endoscope apparatus comprising:
the medical support device according to claim 1; and
the camera.

19. A medical support method comprising:
acquiring a plurality of frames obtained in time series by imaging, with a camera, an inside of a luminal organ into which a hemostat is inserted, the plurality of frames showing stages of a hemostasis treatment using the hemostat; and
outputting hemostasis period identification information that enables determination that a period from when the stages shown in the plurality of acquired frames change from a hemostasis preparation stage using the hemostat to a hemostasis start stage using the hemostat until a certain period of time elapses is a hemostasis period during which the hemostasis treatment is performed.

20. A non-transitory computer-readable storage medium storing a program executable by a computer to execute processing including:
acquiring a plurality of frames obtained in time series by imaging, with a camera, an inside of a luminal organ into which a hemostat is inserted, the plurality of frames showing stages of a hemostasis treatment using the hemostat; and
outputting hemostasis period identification information that enables determination that a period from when the stages shown in the plurality of acquired frames change from a hemostasis preparation stage using the hemostat to a hemostasis start stage using the hemostat until a certain period of time elapses is a hemostasis period during which the hemostasis treatment is performed.
